(19) United States
(12) Patent Application Publication
(10) Pub. No.: US 2020/0237632 A1
(43) Pub. Date: Jul. 30, 2020

(54) COMPOSITIONS HAVING ENHANCED UV PROTECTION PROPERTIES AND METHODS OF MAKING SAME

(71) Applicant: Presperse Corporation, Somerset, NJ (US)

(72) Inventors: Zsolt Szabados, White Plains, NY (US); Kristina Kannheiser Mazzola, Brick, NJ (US)

(21) Appl. No.: 16/773,075

(22) Filed: Jan. 27, 2020

Related U.S. Application Data
(60) Provisional application No. 62/796,910, filed on Jan. 25, 2019.

(51) Int. Cl. A61K 8/25; A61K 8/02; A61Q 17/04; A61K 8/27; A61K 8/29; A61K 8/35; A61K 8/37; A61K 8/41

(52) U.S. Cl. CPC A61K 8/25; A61K 8/025; A61Q 17/04; A61K 8/27; A61K 8/29; A61K 2800/624; A61K 8/37; A61K 8/41; A61K 2800/48; A61K 2800/412; A61K 8/35

(57) ABSTRACT

The present invention relates to compositions with enhanced UV screening properties and methods of making same, and specifically, UV screening compositions including UV filters and silica boosters. The compositions consist of any suitable cosmetically acceptable carrier, one or more organic or inorganic UV filters and spherical silica. The present invention also discloses methods of making UV-screening compositions including spherical silica, wherein the methods comprise adding spherical silica during the process of combining the cosmetically acceptable carrier and UV filters or after the cosmetically acceptable carrier and UV filters have been combined.

### CROSS-REFERENCE TO RELATED APPLICATION

This application claims the priority of U.S. Provisional Patent Application No. 62/796,910, filed Jan. 25, 2019, the content of which is hereby incorporated by reference in its entirety.

### FIELD OF THE INVENTION

The invention relates to compositions with enhanced UV screening properties and methods of making same, and specifically, UV screening compositions including UV filters and silica boosters and methods of making same.

### BACKGROUND

It is estimated that one in five Americans will develop skin cancer in their lifetime. Many such cancers are attributable to exposure from harmful ultraviolet (UV) radiation. Sunscreen can help prevent skin cancer by providing protection from harmful UVA and UVB solar radiation. The degree to which a given sunscreen can protect the skin against UVB and UVA rays is generally designated by its sunscreen protection factor (SPF) as well as critical wavelength (CW). The SPF is a relative measure of how long a sunscreen will protect skin from reddening attributable to UVB and UVA rays. For example, SPF30 means the sunscreen will screen about 97% of UVB and UVA rays. However, even sunscreens with a very high SPF cannot screen UVA rays unless they include a UV filter capable of screening UVA rays. Therefore, it is generally recommended that for adequate protection from both UVA and UVB rays a sunscreen having an SPF of at least 15 and one or more UVA filters be used.

### SUMMARY OF THE INVENTION

In one or more embodiments, UV-screening compositions including spherical silica boosters are disclosed. The spherical silica boosters increase the attenuation of UV filters in the compositions, thereby increasing the SPF level.

Compositions may include any suitable cosmetically acceptable carrier, one or more organic or inorganic UV filters and spherical silica.

In one or more embodiments, a composition includes a cosmetically acceptable carrier, an organic UV filter and spherical silica.

In a further embodiment, a composition includes a cosmetically acceptable carrier, an inorganic UV filter and spherical silica.

In still further embodiments, methods are disclosed for making UV-screening compositions including spherical silica. In one or more embodiments, a method is disclosed which involves adding spherical silica during the process of combining the cosmetically acceptable carrier and UV filters. In still further embodiments, a method is disclosed which involves adding spherical silica after the cosmetically acceptable carrier and UV filters have been combined.

### BRIEF DESCRIPTION OF THE DRAWINGS

So that those having ordinary skill in the art will have a better understanding of how to use the disclosed and methods, reference is made to the accompanying figures wherein:

FIG. 1 is a graphical depiction of absorbance for formulations including inorganic UV filters containing 1% uncoated silica booster material compared to a control according to one or more embodiments of the present disclosure;

FIG. 2 is a graphical depiction of absorbance for formulations including inorganic UV filters containing 3% uncoated silica booster material compared to a control according to one or more embodiments of the present disclosure;

FIG. 3 is a graphical depiction of absorbance for formulations including organic UV filters containing 1% uncoated silica booster material compared to a control according to one or more embodiments of the present disclosure;

FIG. 4 is a graphical depiction of absorbance for formulations including organic UV filters containing 3% uncoated silica booster material compared to a control according to one or more embodiments of the present disclosure;

FIG. 5 is a is a graphical depiction of absorbance for formulations including inorganic UV filters containing 1-3% coated silica booster material compared to a control according to one or more embodiments of the present disclosure;

FIG. 6 is a is a graphical depiction of absorbance for formulations including inorganic UV filters containing 1-3% uncoated, porous silica silica booster material compared to a control according to one or more embodiments of the present disclosure;

FIG. 7 is a is a graphical depiction of absorbance for formulations including inorganic UV filters containing 1-3% uncoated, non-porous booster material compared to a control according to one or more embodiments of the present disclosure; and

FIG. 8 is a is a graphical depiction of absorbance for formulations including inorganic UV filters containing 1-3% uncoated, non-porous silica booster material compared to a control according to one or more embodiments of the present disclosure.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

UV-screening compositions are disclosed which may include any suitable cosmetically acceptable carrier, one or more organic or inorganic UV filters and a UV booster.

The cosmetically acceptable carrier is preferably present in the composition in an amount from 55-90 weight percent, more preferably from 77-82 weight percent, and may include an emulsifier, humectant, emollient and a thickener. The carrier may also include one or more further components such as but not limited to preservatives, chelating agents, dyes, pigments, diluents, vitamins, fragrance, etc.

Suitable emulsifiers include but are not limited to glyceryl esters, glutamate derivatives, C8-C20 glycosides, polyethylene glycol and derivatives thereof such as but not limited to polyethylene glycol ethers of fatty alcohols, polypropylene glycol and derivatives thereof, alkyl sulfates, amino acid salts, esters and acyl derivatives, glycosaminoglycans and derivatives thereof, borax with beeswax, beeswax, BTMS 25%, carbomer, cetaryl alcohol, emulsifying wax-NF, lecithin, PEG-20 stearate, propylene glycol, emulsifying wax, stearyl alcohol NF, polysorbate 80, etc.

Suitable humectants include but are not limited to diols, triols and polyols, such as glycerin, phytantriol, erythritol, propylene glycol, butylene glycol, 1,2,6-hexanetriol, triethyelene glycol, sorbitol, PEG, sodium PCA, etc.

Suitable emollients include but are not limited to liquid and solid emollients, including for example liquid emollients such as hydrocarbons (C3-C16 linear and isomers thereof) and polymers thereof, and esters (C6-C34, branched or linear) such as carbonates, isononanoates, caprylates, oleates, adipates, stearates, myristates, laurates, benzoates, maleates, pentanoates, hexanoates and heptanoates. Solid emollients include but are not limited to C12-C20 fatty alcohols such as cetyl-, stearyl-, cetearyl-, behenyl-, and fatty esters such as distearates, palmitates, myristates, stearates, laurates, etc.

Suitable thickeners include but are not limited to naturally-derived thickeners such as polysaccharide thickeners, e.g., corn sugar, cellulose, agar, carrageenan, starches; lipid thickeners such as but not limited to cetyl alcohol, stearyl alcohol, carnauba wax, and stearic acid; mineral thickeners including but not limited to inorganic salts (e.g., silicates) and inorganic clays (e.g. bentonite and derivatives thereof); and synthetic thickeners including but not limited to acrylates such as carbomers, co- and cross-polymers and salts thereof.

Table 1 provides non-limiting, exemplary components for the carrier.

TABLE 1
Exemplary Cosmetic Carrier

| | | weight % in |
|---|---|---|
| Component | Non-limiting Examples | carrier |
| Emulsifier | Glyceryl Esters, Glutamate derivatives, | 0.01-10% |
| | Glycoside (C8-C20), Polyethylene Glycol | |
| | based and its derivatives, Polypropylene | |
| | Glycol based and its derivatives | |
| Humectant | Diols, triols, polyols | 1.0-10.0% |
| Emollient, | Hydrocarbon (C3-C16) and polymers | 2.0-20.0% |
| liquid | thereof, Esters (C6-C34, branched | |
| | or linear) | |
| Emollient, | Fatty Alcohol (C12-C18), fatty esters | 0.01-10.0% |
| solid | | |
| Thickener | Polysaccharide, Inorganic Salts, Inorganic | 0.01-10% |
| | Clays | |
| Preservative | Any cosmetically acceptable preservative | 0-1.0% |
| Chelating | EDTA and its derivatives | 0-1.0% |
| Agent | | |
| Water | | 40-90% |

Suitable inorganic UV filters may be present in the composition from 1-50 weight percent and may include one or more of titanium dioxide (coated or non-coated) and zinc oxide (coated or non-coated). In one or more embodiments, the composition may include 1-25% w/w titanium dioxide or 1-25% w/w zinc oxide (w/w refers to the percentage weight of the ingredient or compound in the composition.) In one or more embodiments, the inorganic UV filter of the composition is a combination of titanium dioxide and zinc oxide in a preferred amount of 1-25 weight percent in the composition.

Suitable organic UV filters may be present in the composition from 1 to 18 weight percent and may include one or more of avobenzone, octocrylene and ethylhexyl salicylate. In one or more embodiments the composition includes 1-3% w/w avobenzone, 1-10% w/w octocrylene and/or 2-5% w/w ethylhexyl salicylate. In one or more embodiments the organic UV filter of the composition is a combination of avobenzone, octocrylene and ethylhexyl salicylate.

The UV booster of the composition is spherical silica. The spherical silica may be coated or uncoated. The particle size ranges from 1-25 m. The spherical silica particles may be, but are not limited to, porous, non-porous, or hollow spheres. The oil absorption of the spherical silica may range from 5-300 ml/100 g. In one or more embodiments the composition includes from 0.05-15% w/w spherical silica. Uncoated, porous and hollow sphere spherical silica materials are available commercially for example from JGC Catalysts and Chemicals Ltd. (Japan) under the trade names Spheron BA-1, Spheron LP-230 and Silica Balloon BA-4 respectively. Coated spherical silica having a methicone coating is available commercially for example from Presperse (Somerset, N.J.) under the trade name Spheron P-1500-030.

In some embodiments, the spherical silica may be added “in-process” to either the water or oil phase prior to emulsification. In other embodiments the spherical silica may be added to the formulation after emulsification.

### EXAMPLES AND EXPERIMENTS

The following are non-limiting, exemplary formulations in accordance with the present disclosure.

### Example 1—UV Booster Added after Emulsification (“Post Addition”)

TABLE 2
Post-addition Formulations

| Phase | Trade Name | INCI Name | w/w % |
|---|---|---|---|
| A | Water | Water | balance |
| A | Trilon BD | Disodium EDTA | 0.05 |
| A | Glycerin | Glycerin | 2.50 |
| A | Pellicer L-30 | Sodium Dilauramidoglutamide | 0.20 |
| | | Lysine | |
| A | Eumulgin SG | Sodium Stearoyl Glutamate | 0.50 |
| A | Keltrol CG-T | Xanthan Gum | 0.50 |
| B | Emulgade | Cetearyl Glucoside (and) | 3.50 |
| | PL 68/50 | Cetearyl Alcohol | |
| B | Jeechem CS-50 | Cetearyl Alcohol | 1.50 |
| B | Jeechem DIA | Diisopropyl Adipate | 5.00 |
| B | Jeechem CTG | Caprylic/Capric Triglyceride | 10.00 |
| C | Various | UV Filters | 17.00 or 20.00 |
| D | Euxyl PE9010 | Phenoxyethanol (and) | 1.00 |
| | | Ethylhexylglycerin | |
| E | Various | Silica or Silica (and) Methicone | 1.00 or 3.00 |

Four versions of a post-addition method composition were made in accordance with Table 2. Version 1 employed a) 20% w/w of an inorganic UV filter, which included 10% w/w titanium dioxide coated with aluminum hydroxide (and) hydrated silica (and) dimethicone/methicone copolymer (MTY-100SAS, available from Presperse, Somerset, N.J.) and 10% w/w zinc oxide coated with triethoxycaprylysilane (MZX-3040TS, Presperse, Somerset, N.J.); and b) 1% w/w uncoated or coated spherical silica. Version 2 employed a) the same inorganic UV filter as Version 1 and b) 3% w/w uncoated or coated spherical silica.

Version 3 employed a) 17% w/w of an organic UV filter, which including 10% w/w octocrylene (Uvinul® N 539 T, BASF), 5% w/w ethylhexyl salicylate (Neo Heliopan® OS, Symrise) and 2% w/w avobenzone (Neo Heliopan® 357, Symrise) and b) 1% w/w uncoated or coated spherical silica. Version 4 employed a) the same organic UV filter as Version 3 and b) 3% w/w uncoated or coated spherical silica.

All versions of the compositions were made using the following method:

1. Weigh out part A (water phase of the carrier) in a side beaker and heat to 75-85° C. (75° C. preferred for steps 1-5).
2. Weigh out part B (oil phase of the carrier) in main beaker, heat and mix to 75-85° C.
3. Disperse Part C (UV filters) into Part B at 75-85° C., homogenize until all clumps are dispersed.
4. Add part BC to A at 75-85° C. under homogenizer, homogenize for approximately 5 minutes at −8,000 rpm, which leads to the formation of an emulsion.
5. Switch back to mixer and add part D while the temperature is still raised at 75-85° C.
6. Continue to mix, cool to room temperature.
7. Add silica (E) under propeller mixing.

### Example 2—UV Booster Added “in-Process”

TABLE 3
In-process Formulations

| Phase | Trade Name | INCI Name | w/w % |
|---|---|---|---|
| A | Water | Water | balance |
| A | Trilon BD | Disodium EDTA | 0.05 |
| A | Glycerin | Glycerin | 2.50 |
| A | Pellicer L-30 | Sodium Dilauramidoglutamide | 0.20 |
| | | Lysine | |
| A | Eumulgin SG | Sodium Stearoyl Glutamate | 0.50 |
| A | Keltrol CG-T | Xanthan Gum | 0.50 |
| B | Emulgade | Cetearyl Glucoside (and) | 3.50 |
| | PL 68/50 | Cetearyl Alcohol | |
| B | Jeechem CS-50 | Cetearyl Alcohol | 1.50 |
| B | Jeechem DIA | Diisopropyl Adipate | 5.00 |
| B | Jeechem CTG | Caprylic/Capric Triglyceride | 10.00 |
| C | Various | UV Filters | 17.00 or 20.00 |
| D | Euxyl PE9010 | Phenoxyethanol (and) | 1.00 |
| | | Ethylhexylglycerin | |
| B | Various | Silica or Silica (and) Methicone | 1.00 or 3.00 |

Four versions of an “in-process” method composition were made in accordance with Table 3. Version 1 employed a) 20% w/w of an inorganic UV filter, which included 10% w/w titanium dioxide coated with aluminum hydroxide (and) hydrated silica (and) dimethicone/methicone copolymer (MTY-100SAS, available from Presperse, Somerset, N.J.) and 10% w/w zinc oxide coated with triethoxycaprylysilane (MZX-3040TS, Presperse, Somerset, N.J.); and b) 1% w/w uncoated or coated spherical silica. Version 2 employed a) the same inorganic UV filter as Version 1 and b) 3% w/w uncoated or coated spherical silica.

Version 3 employed a) 17% w/w of an organic UV filter, which included 10% w/w octocrylene (Uvinul® N 539 T, BASF), 5% w/w ethylhexyl salicylate (Neo Heliopan® OS, Symrise) and 2% w/w avobenzone (Neo Heliopan® 357, Symrise); and b) 1% w/w uncoated or coated spherical silica. Version 4 employed a) the same organic UV filter as Version 3 and b) 3% w/w uncoated or coated spherical silica.

All versions of the compositions were made using the following method:

1. Weigh out part A (water phase of the carrier) in a side beaker and heat to 75-85° C. (75° C. preferred for steps 1-5).
2. Weigh out part B (oil phase of the carrier+silica) in main beaker, heat and mix to 75-85° C.
3. Disperse Part C (UV filters) into Part B at 75-85° C., homogenize until all clumps are dispersed.
4. Add part BC to A at 75-85° C. under homogenizer, homogenize for approximately 5 minutes at −8,000 rpm, which leads to the formation of an emulsion.
5. Switch back to mixer and add part D while the temperature is still raised at 75-85° C.
6. Continue to mix, cool to room temperature.

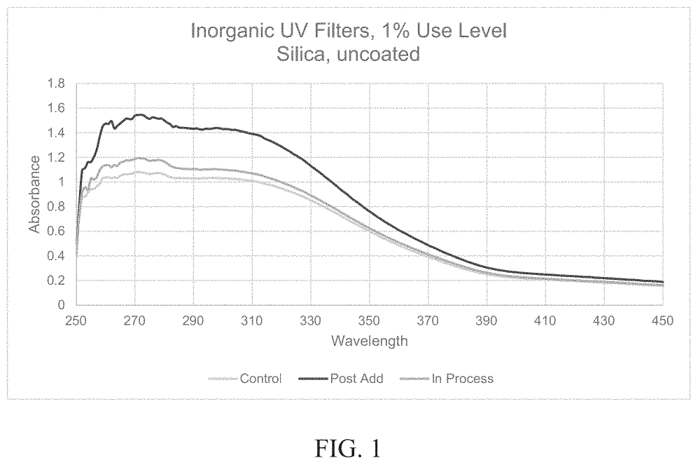

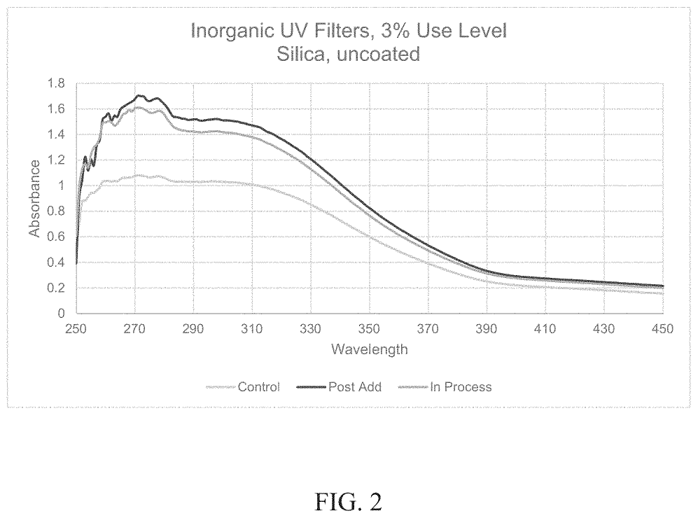

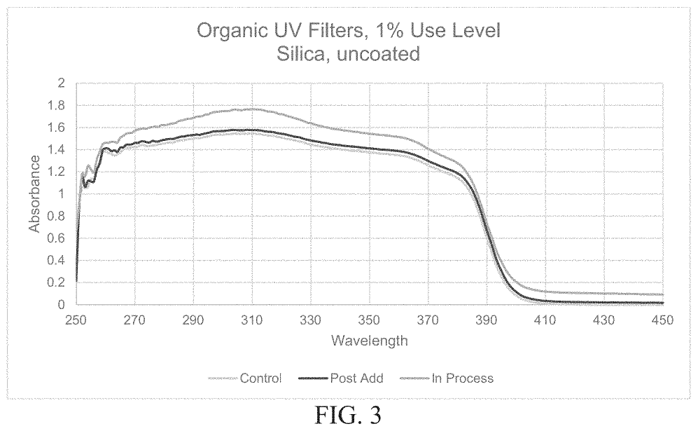

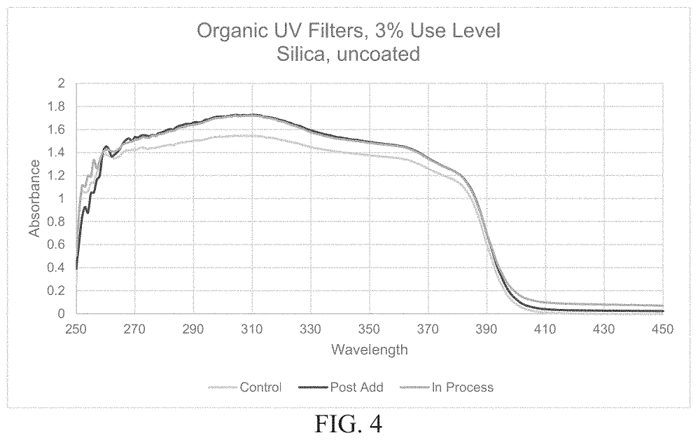

Now referring to FIGS. 1-4 and Tables 4-5, compositions employing spherical silica made in accordance with Examples 1 and 2 were evaluated. SPF testing employed a LabSphere VS2000, using molded PMMA plates with a film thickness of 2 mg/cm². Critical Wavelength testing employed a LabSphere VS2000 using molded PMMA plates with a film thickness of 0.75 mg/cm².

TABLE 4
Percentage of Boost, SPF and Opacity for Inorganic Formulations

| Process | Percent Boosted | SPF | Opacity |
|---|---|---|---|
| Control | — | 9.9 | 8.7 |
| Post Add - 1% | 135% | 23.3 | 13.8 |
| In Process - 1% | 17% | 11.6 | 8.7 |
| Post Add - 3% | 180% | 27.7 | 14.6 |
| In Process - 3% | 106% | 20.4 | 10.8 |

TABLE 5
Percentage of Boost, SPF and Opacity for Organic Formulations

| Process | Percent Boosted | SPF | Opacity |
|---|---|---|---|
| Control | — | 35.0 | 1.4 |
| Post Add - 1% | 3% | 36.0 | 1.7 |
| In Process - 1% | 64% | 57.3 | 1.8 |
| Post Add - 3% | 48% | 51.9 | 3.6 |
| In Process - 3% | 48% | 51.9 | 3.5 |

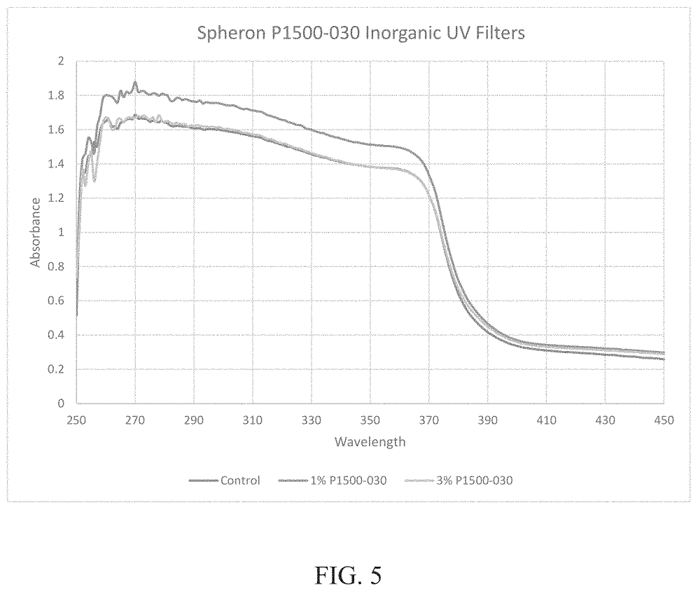

Now referring to FIGS. 5-8 and Tables 6-13, compositions made in accordance with Example 1 (“post-addition”), Versions 1 and 2 employing various forms of spherical silica were evaluated. SPF testing employed a LabSphere VS2000, using molded PMMA plates with a film thickness of 2 mg/cm². Critical Wavelength testing employed a LabSphere VS2000 using molded PMMA plates with a film thickness of 0.75 mg/cm².

With reference to FIG. 5 and Table 7, formulations containing the coated spherical silica of Table 6 were evaluated.

TABLE 6
Coated Spherical Silica - Post Addition

| Trade | | INCI | Particle | Oil | | |
|---|---|---|---|---|---|---|
| Name | Supplier | Name | Size | Absorption | Morphology | Coating |
| Spheron | Presperse | Silica (and) | 9.3 μm | 55 mL/100 g | Non-porous | Methicone |
| P1500-030 | | Methicone | | | | |

TABLE 7
Results - Formulations Using Inorganic UV
Filters and Spherical Silica of Table 6

| Process | Percent Boosted | SPF |
|---|---|---|
| Control | — | 35 |
| Post Add - 1% | 77% | 62 |
| Post Add - 3% | 34% | 47 |

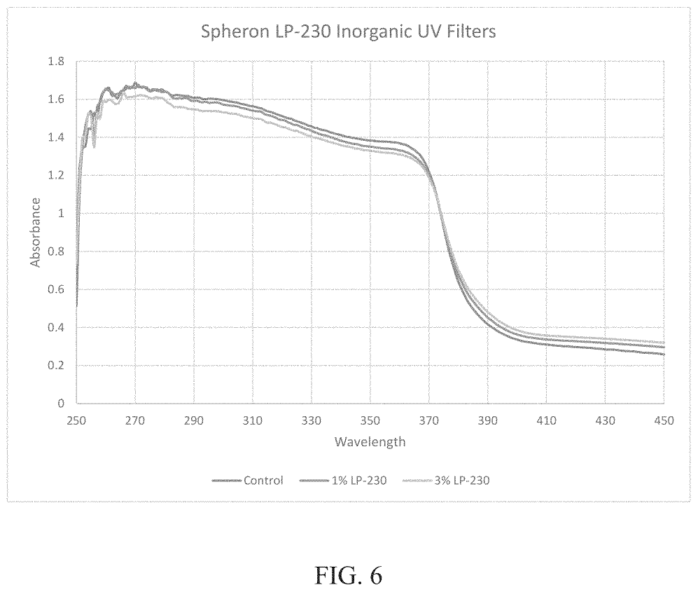

With reference to FIG. 6 and Table 9, formulations containing the uncoated spherical silica of Table 8 were evaluated.

TABLE 8
Uncoated, Porous Large Spherical Silica - Post Addition

| Trade | | INCI | Particle | Oil | | |
|---|---|---|---|---|---|---|
| Name | Supplier | Name | Size | Absorption | Morphology | Coating |
| Spheron | JGC&C | Silica | 13.9 μm | 230 mL/ | Porous, | None |
| LP-230 | | | | 100 g | Large | |

TABLE 9
Results - Formulations Using Inorganic UV
Filters and Spherical Silica of Table 8

| Process | Percent Boosted | SPF |
|---|---|---|
| Control | — | 35 |
| Post Add - 1% | 23% | 43 |
| Post Add - 3% | 0% | 35 |

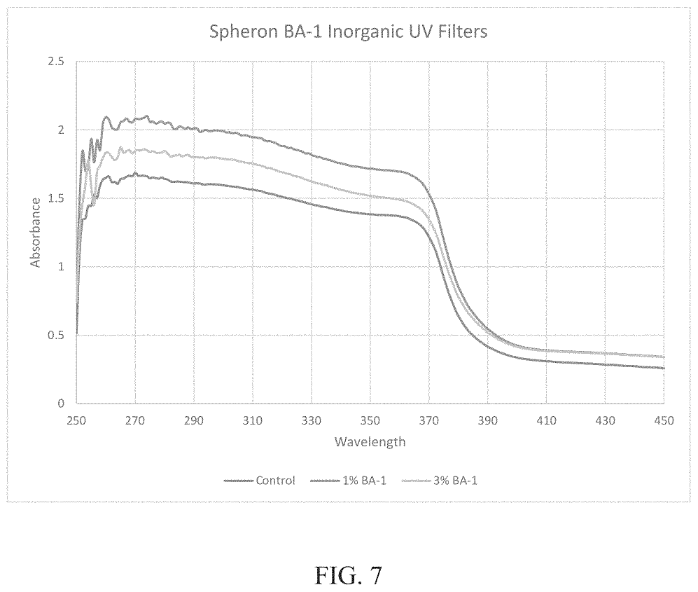

With reference to FIG. 7 and Table 11, formulations containing the uncoated spherical silica of Table 10 were evaluated.

TABLE 10
Uncoated, Non-porous Spherical Silica - Post Addition

| Trade | | INCI | Particle | Oil | | |
|---|---|---|---|---|---|---|
| Name | Supplier | Name | Size | Absorption | Morphology | Coating |
| Spheron | JGC&C | Silica | 13.7 μm | 50 mL/ | Non-porous | None |
| BA-1 | | | | 100 g | | |

TABLE 11
Results - Formulations Using Inorganic UV
Filters and Spherical Silica of Table 10

| Process | Percent Boosted | SPF |
|---|---|---|
| Control | — | 35 |
| Post Add - 1% | 160% | 91 |
| Post Add - 3% | 64% | 83 |

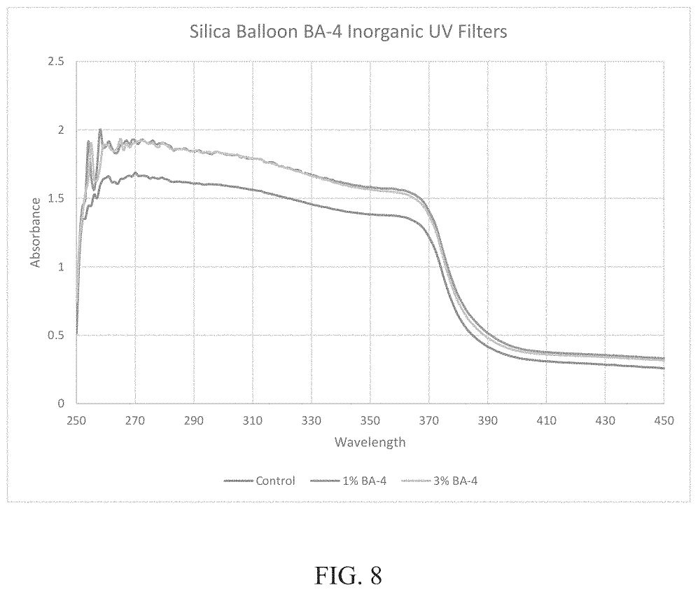

With reference to FIG. 8 and Table 13, formulations containing the uncoated spherical silica of Table 12 were evaluated.

TABLE 12
Uncoated, Non-porous Spherical Silica Balloon - Post Addition

| Trade | | INCI | Particle | Oil | | |
|---|---|---|---|---|---|---|
| Name | Supplier | Name | Size | Absorption | Morphology | Coating |
| Silica | JGC&C | Silica | 3.5 μm | 40 mL/ | Non-porous | None |
| Balloon | | | | 100 g | | |
| BA-4 | | | | | | |

TABLE 13
Results - Formulations Using Inorganic UV
Filters and Silica Balloon of Table 10

| Process | Percent Boosted | SPF |
|---|---|---|
| Control | — | 35 |
| Post Add - 1% | 114% | 75 |
| Post Add - 3% | 77% | 62 |

Although the methods and materials of the present disclosure have been described with reference to exemplary embodiments thereof, the present disclosure is not limited thereby. Indeed, the exemplary embodiments are implementations of the disclosed compositions and systems are provided for illustrative and non-limitative purposes. Changes, modifications, enhancements and/or refinements to the disclosed compositions and systems may be made without departing from the spirit or scope of the present disclosure. Accordingly, such changes, modifications, enhancements and/or refinements are encompassed within the scope of the present invention.

1. A UV screening composition comprising a cosmetically acceptable carrier, at least one UV filter selected from the group consisting of inorganic UV filters and organic UV filters; and a UV booster comprising a spherical silica, wherein the spherical silica has a particle size of from 1 to 25 μm and an oil absorption value of from 5 to 300 ml per 100 grams.

2. The composition of claim 1, wherein the carrier includes one or more compounds selected from the group consisting of emulsifiers, humectants, emollients and thickeners.

3. The composition of claim 2, wherein the carrier further includes one or more compounds selected from the group consisting of preservatives, chelating agents, dyes, pigments, diluents, vitamins and fragrances.

4. The composition of claim 2, wherein the carrier comprises 0.01 to 10.0 weight percent emulsifier, 1.0 to 10.0 weight percent humectant, 2.0 to 20.0 weight percent liquid emollient, 0.01 to 10.0 weight percent solid emollient, 0.01 to 10 weight percent thickener and 40 to 90 weight percent water, the weight percents based on the overall weight of the carrier.

5. The composition of claim 1, wherein the inorganic UV filters are selected from the group consisting of titanium dioxide and zinc oxide.

6. The composition of claim 5, wherein the inorganic UV filters are present in the composition from 1 to 50 weight percent.

7. The composition of claim 1, wherein the organic filters are selected from the group consisting of avobenzene, octocrylene and ethylhexyl salicylate.

8. The composition of claim 7, wherein the composition includes avobenzene from 1 to 3 weight percent, octocrylene from 1 to 10 weight percent and ethylhexyl salicylate from 2 to 5 weight percent.

9. (canceled)

10. The composition of claim 1, wherein the spherical silica is present in the composition from 0.05 to 15 weight percent.

11. The composition of claim 1, wherein the spherical silica are coated with a methicone coating.

12. The composition of claim 4, wherein the inorganic UV filters are selected from the group consisting of titanium dioxide and zinc oxide and wherein the inorganic UV filters are present in the composition from 1 to 50 weight percent.

13. The composition of claim 4, wherein the organic UV filters are selected from the group consisting of avobenzene from 1 to 3 weight percent in the composition, octocrylene from 1 to 10 weight percent in the composition and ethylhexyl salicylate from 2 to 5 weight percent in the composition.

14. The composition of claim 1, wherein the inorganic UV filters are a combination of titanium dioxide and zinc oxide and are present in the composition from 1 to 25 weight percent and the spherical silica are present in the composition from 0.05 to 15 weight percent.

15. The composition of claim 1, wherein the organic UV filters are avobenzene from 1 to 3 weight percent in the composition, octocrylene from 1 to 10 weight percent in the composition and ethylhexyl salicylate from 2 to 5 weight percent in the composition; and the spherical silica are present in the composition from 0.05 to 15 weight percent.

16.-21. (canceled)
